(19) United States
(12) Patent Application Publication
(10) Pub. No.: US 2014/0342385 A1
(43) Pub. Date: Nov. 20, 2014

(54) MEDIUM AND METHOD FOR DETECTING PATHOGENIC YERSINIA ENTEROCOLITICA BACTERIA

(71) Applicant: Alain RAMBACH, Paris (FR)

(72) Inventor: Alain Rambach, Paris (FR)

(21) Appl. No.: 14/360,796

(22) PCT Filed: Nov. 28, 2012
(86) PCT No.: PCT/EP2012/073802

§ 371 Date: May 27, 2014

(30) Foreign Application Priority Data
Nov. 28, 2011 (FR) 1160876

(51) Int. Cl. C12Q 1/34

(52) U.S. Cl. CPC C12Q 1/34
USPC 435/18

(57) ABSTRACT

The present invention relates to a culture medium for detecting pathogenic Yersinia enterocolitica bacteria, comprising at least one chromogenic agent which is a substrate of an acetyl-glucosaminidase, to a method of detection using such a medium and to the use of such a medium for the detection of pathogenic Yersinia enterocolitica bacteria.

This invention relates to a culture medium for the detection of pathogenic Yersinia enterocolitica bacteria comprising at least one chromogenic agent substrate of an acetyl-glucosaminidase, a detection method using such a medium and the use of such a medium for detection of pathogenic Yersinia enterocolitica bacteria.

Yersinia are enterobacteria currently composed of three species containing pathogenic strains for animal and for man, namely Yersinia pestis, Yersinia enterocolitica, and Yersinia Pseudotuberculosis, and species that are normally non-pathogenic, Yersinia frederiksenii, Yersinia kristensenii, Yersinia intermedia, Yersinia aldovae, Yersinia mollaretii, Yersinia bercovieri and Yersinia rodhei.

Yersinia enterocolitica is responsible for enterocolitis. It is usually reported in children and its symptoms are diarrhoea, fever, abdominal pain and sometimes vomiting.

Research for Yersinia has lead to the development of many culture media. These media usually incubated at a temperature of 30° C., particularly to preserve, express and preserve the pathogenic character, have led to the development of methods for characterising pathogenic strains, for example by identifying a reduction in growth on calcium-deficient media and a more intense colouring by crystal violet at the higher temperature of 37° C., abnormal for this species.

Isolation media according to prior art include the CIN agar medium (Schiemann D A, Synthesis of a selective agar medium for Yersinia enterocolitica, Can. J Microbiol, 1979) that uses the mannitol character revealed by a change in the pH and a coloured indicator, the CAL agar medium (Dudley et al., Medium for isolation of Yersinia enterocolitica, Journal of Clinical Biology, 1979) that uses the cellobiose character revealed by a change in the pH and a coloured indicator, for detecting Yersinia.

Furthermore, a VYE agar medium that uses the esculine character to distinguish non-pathogens by a black halo diffusing around colonies, has also been produced (Fukushima, New Selective Agar Medium for Isolation of Virulent Yersinia enterocolitica, Journal of Clinical Biology, 1987). Weagant has developed a medium derived from CIN Agar by replacing mannitol by cellobiose and adding X-glucoside (5-bromo-4-chloro-3-indoyl beta-D-glucopyranoside) (Weagant, A new chromogenic agar medium for detection of potentially virulent Yersinia enterocolitica, Elsevier, 2008).

Garcia-Aguayo et al. developed a Xylose-Galactosidase medium with 5-bromo 6-chloro-3-indoxyl-beta-galactoside in 1999 that uses the beta-galactosidase character (Garcia-Aguayo et al., Evaluation of Xylose-Galactosidase Medium, a new Plate for the isolation of Salmonella, Shigella, Yersinia and Aeromonas Species, Eur J Clin Microbio Infect Dis., Volume 18, 1999).

However, such media cannot give sufficient sensitivity and also lead to false positives that limit the reliability of the results.

Therefore it is important to have tools and methods for the detection of these bacteria that combine good sensitivity and good specificity and particularly ease of use in order to be able to simplify tests as much as possible, to do them quickly and in large numbers, or even to automate them, in order to test food and/or hospital hygiene, while quickly differentiating between different pathogenic strains of Yersinia enterocolitica.

Therefore there is a real need to have a simpler, more specific, more direct and less expensive detection technique capable of differentiating different strains of pathogenic Yersinia enterocolitica bacteria, while avoiding the combination of several tests increasing the time necessary to obtain results and increasing the risk of parasite contaminations or mistakes, and the risk of diffusion of the bacteria.

Media including indoxyl-beta-galactosides were tested. Colouring of Yersinia enterocolitica has been observed, but it occurs slowly. Colouring also developed on a very large number of strains of non-Yersinia species.

Surprisingly and unexpectedly, the Inventor has demonstrated that the use of chromogenic substrates of hexosaminidase, and particularly acetyl-glucosaminidase, allows to isolate quickly, sensitively and specifically pathogenic Yersinia enterocolitica bacteria. In particular, when it is used on a solid gelose medium, the detection method developed by the Inventor can be done directly, for example from a food sample or from a patient, without requiring a preliminary step to isolate the different strains present in this sample.

Therefore one purpose of this invention is a culture medium for the detection of pathogenic Yersinia enterocolitica bacteria comprising:
nutrients necessary for growth of said bacteria to be detected, and
at least one chromogenic agent substrate of an acetyl-glucosaminidase.

“Culture medium” means a medium enabling growth of said bacteria to be detected. Said culture medium provides the nutrients necessary for growth of said bacteria to be detected.

“Pathogenic Yersinia enterocolitica bacteria” means Yersinia enterocolitica bacteria with a biotype chosen from the group composed of biotypes IB, 2, 3, 4 and 5. Biotype 1A of Yersinia enterocolitica is frequent and is not recognised as pathogenic for man and animals.

“Nutrients necessary for growth of said bacteria to be detected” means the composition of a basic medium necessary for said growth. Those skilled in the art will be perfectly familiar with the composition of such media and would be capable of adapting it if necessary according to the specificity of specific micro-organisms or to constraints that could be related to some cases of this invention (for example transparency of the medium). These nutrients are chosen particularly from the group comprising carbon, nitrogen, sulphur, phosphorus, vitamins, growth inducers, carbon hydrates, salts (for example calcium, magnesium, manganese, sodium, potassium), nutrient complexes (for example amino acids, blood, serum, albumin) and peptones and extracts from animal and plant tissues.

The culture medium used for the purposes of this invention may be in solid, semi-solid, liquid or freeze dried form. Preferably, said culture medium is a gelose medium that may for example be based on agar. Culture medium presentations that can be used include Petri boxes on which micro-organisms develop.

“Chromogenic agent” or “chromogenic substrate” refers to a compound carrying a chromophore released after hydrolysis by a specific enzyme. The chromophore thus released gives its colour to colonies including said enzyme. Preferably, said chromophore substrate is a precipitating chromophore.

According to this invention, acetyl-glucosaminidase refers to an enzyme capable of releasing an N-acetyl-D-glucosamine residue by hydrolysis.

The chromogenic substrate is preferably used at a concentration of between 0.01 and 0.5 g/l, and preferably between 0.05 and 0.2 g/l. One particularly preferred concentration is about 0.1 g/l.

Preferably, said chromophore is chosen from the group composed of derivatives of indoxyl, halogeno-indoxyl (bromo-indoxyl, chloro-indoxyl, fluoro-indoxyl, iodo-indoxyl, dichloro-indoxyl, chloro-bromo-indoxyl, tri-chloro-indoxyl), methyl-indoxyl and hydroxy-quinoline. Particularly preferred derivatives are chosen from the group composed of the following derivatives: 6-chloro-indoxyl, 5-bromo-indoxyl, 3-bromo indoxyl, 6-fluoro-indoxyl, 5-iodo-indoxyl, 4,6-dichloro-indoxyl, 6,7-dichloro-indoxyl, 5-bromo-4-chloro-indoxyl, 5-bromo-6-indoxyl, 4,6,7-trichloro-indoxyl, N-methyl-indoxyl and 8-hydroxy-quinoline.

Advantageously, the chromogenic substrate of said acetyl-glucosaminidase is an indoxyl-beta-glucosaminide, preferably chosen from the group composed of 5-bromo-6-chloro-3-indoxyl-N-acetyl-beta-D-glucosaminide, 6-chloro-3-indolyl-N-acetyl-beta-D-glucosaminide and 6-fluoro-indoxyl-N-acetyl-beta-D-glucosaminide, and more particularly 5-bromo-6-chloro-3-indoxyl-N-acetyl-beta-D-glucosaminide.

Alternatively, said acetyl-glucosaminidase uses an indolyl-beta-glucosaminide, and preferably 5-bromo-4-chloro-3-indolyl-N-acetyl-beta-D-glucosamnide as its chromogenic substrate.

In one particular preferred embodiment, the medium according to this invention further comprises a chromogenic agent substrate of beta-glucosidase, preferably said chromogenic agent releasing after hydrolysis a chromophore with a colour distinct from the chromophore that might be released after hydrolysis of the chromogenic agent substrate of an acetyl-glucosaminidase.

Preferably, said chromogenic agent substrate of beta-glucosidase is 5-bromo-4-chloro-3-indolyl-beta-D-glucoside (also called X-glucoside) when the chromogenic agent substrate of acetyl-glucosaminidase of the medium according to the invention is chosen from the group composed of 5-bromo-6-chloro-3-indoxyl-N-acetyl-beta-D-glucosaminide, 6-chloro-3-indolyl-N-acetyl-beta-D-glucosaminide and 6-fluoro-indoxyl-N-acetyl-beta-D-glucosaminide.

Preferably, said chromogenic agent substrate of beta-glucosidase is 8-hydroxy-quinoline-beta-D-glucoside (also called X-glucoside) when the chromogenic agent substrate of acetyl glucosaminidase in the medium according to the invention is 5-bromo-4-chloro-3-indolyl-N-acetyl-beta-D-glucosaminide.

Said chromogenic agent substrate of beta-glucosidase is preferably used at a concentration varying from 0.01 to 0.5 g/l, preferably from 0.05 to 0.2 g/l. One particularly preferred concentration is about 0.1 g/l.

In one particularly preferred embodiment, the medium according to this invention includes 5-bromo-6-chloro-3-indoxyl-N-acetyl-beta-D-glucosaminide and 5-bromo-4-chloro-3-indolyl-beta-D-glucoside.

Those skilled in the art will easily adjust the effective quantity of chromogens in the medium according to this invention, based on their general knowledge and the results described in the following examples.

Culture media according to this invention may possibly contain one or several antimicrobial agents, particularly one or several antibiotics and one or several antifungal agents.

Said antimicrobial agent(s) can limit the growth of micro-organisms other than said at least one specific micro-organism to be detected.

The effective quantity of antimicrobial agent to be used can be determined simply by those skilled in the art based on their general knowledge.

Advantageously, the medium according to this invention further comprises Irgasan.

According to this invention, Irgasan refers to a compound with formula 5-chloro-2-(2,4-dichlorophenoxy)phenol (CAS number: 3380-34-5).

Preferably, the medium according to this invention comprises 5-chloro-2-(2,4-dichlorophenoxy)phenol at a concentration of between 0.0005 and 0.01 g/l, preferably between 0.001 and 0.005 g/l, preferably between 0.001 and 0.003 g/l and even more preferably about 0.002 g/l.

Another aspect of this invention relates to a method for direct detection of pathogenic Yersinia enterocolitica bacteria in a biological sample comprising the following successive steps:

a) inoculating a culture medium as defined above, with said sample,

b) incubating said culture medium under conditions conducive to the growth of pathogenic Yersinia enterocolitica bacteria, and

c) detecting colonies formed on said culture medium corresponding to pathogenic Yersinia enterocolitica bacteria.

Advantageously, the method according to this invention further comprises a step d) to conclude whether or not a particular strain of bacteria is present as a function of the colour of the colonies formed. Consequently, this is a step d) to identify pathogenic Yersinia enterocolitica bacteria in said sample.

“Biological sample” means any type of microbiological sample, such as for example sampling of food materials (dairy products, meat, etc.), sampling of a soil, sampling on a mammal (skin, mucous membranes, etc.), preferably man, or one of its derivatives such as a pre-culture derived from such a sample.

Advantageously, said biological sample is a liquid biological sample such as saliva, blood or urine, a solid biological sample such as faeces or a food product, or a derivative of a liquid or solid biological sample such as pre-culture of such a liquid or solid biological sample.

Also advantageously, said biological sample comprises different micro-organisms that may belong to distinct species or even distinct genera. For example, said biological sample comprises at least two different micro-organisms, preferably at least five different micro-organisms and even more preferably, at least ten different micro-organisms.

“Inoculation” means starting to culture said culture medium with all or some of the biological sample and incubation of said inoculated culture medium.

Those skilled in the art will adapt incubation conditions as a function of the culture medium, the biological sample and the specific micro-organism to be detected as a function of their general knowledge.

The incubation step can be performed at a temperature of between 0° C. and 44° C., preferably between 20° C. and 43° C., more preferably between 28 and 32° C., and even more preferably 30° C.

Preferably, the incubation step lasts for a period of between 16 and 36 hours, and preferably between 18 and 24 hours.

However, depending on the means available to them, those skilled in the art may adapt the temperature and duration of this incubation step based on their general knowledge.

“Direct detection method” means a method that does not include a preliminary step to isolate the different bacterial strains present in the sample, preferably a method that does not include a preliminary step to isolate each bacterial strain present in the sample.

The method according to the invention does not require a step to isolate candidate bacterial colonies on which a more precise confirmation test can be carried out later. Therefore, it is applicable to a raw sample that might include a mix of bacteria.

Preferably, the method according to this invention does not include a preliminary step to isolate the different bacterial strains present in the sample.

Unlike previous methods, the method developed by the Inventor enables fast direct detection of pathogenic Yersinia enterocolitica bacteria.

Another aspect of this invention relates to the use of a culture medium as defined above, for direct detection and differentiation of pathogenic Yersinia enterocolitica bacteria.

The following examples are provided for illustrative purposes and are not intended to limit the scope of this invention.

### EXAMPLE

### Example 1: Comparison of the Medium According to This Invention with a CIN Agar Medium

Becton Dickinson's CIN agar medium used comprises the following elements in g/L: Peptone 17.0, Proteose peptone 3.0, Yeast extract 2.0, D-Mannitol 20.0, Sodium desoxycholate 0.5, Sodium cholate 0.5, Sodium pyruvate 2.0, Heptahydrate magnesium sulphate 0.01, Sodium chloride 1.0, Neutral red 0.03, Crystal Violet 0.001, Irgasan 0.004, Gelose 13.5, Cefsulodine 0.015, Novobiocine 0.0025.

The medium according to this invention includes the following elements in g/L: Peptone 20, Sodium chloride 5, Agar 15, 5-Bromo-4-chloro-3-indolyl-beta-D-glucoside 0.1, 5-bromo-6-chloro-3-indoxyl-N-acetyl-P-D-glucosaminide 0.1, Irgasan 0.002, Tween 80 0.5.

Different bacterial strains are isolated on the medium according to the invention and the CIN Agar medium. The boxes are incubated for 24 h at 30° C.

Table I below presents the results obtained:

TABLE I

| | | | Medium according to | |
|---|---|---|---|---|
| | CIN Agar | | the invention | |
| | Colour | Detection | Colour | Detection |
| Y. enterocolitica | RED | + | MAUVE | + |
| AR5580 | | | | |
| Non pathogenic | RED | + | BLUE | − |
| Y. enterocolitica | | | | |
| 2B154 | | | | |
| Non pathogenic | RED | + | BLUE | − |
| Y. enterocolitica | | | | |
| AR5685 | | | | |
| Citrobacter | RED | + | BLUE | − |
| AR3378 | | | | |
| Citrobacter | RED | + | BLUE | − |
| AR3870 | | | | |
| Citrobacter | RED | + | BLUE | − |
| AR3871 | | | | |
| Citrobacter | RED | + | BLUE | − |
| AR 3030 | | | | |
| Serattia | RED | + | BLUE | − |
| AR 5568 | | | | |
| | | | | − |
| E. coli AR 3741 | NC | − | NC | − |
| Hafnia AR 3862 | NC | − | NC | − |
| Enterobacter AR | NC | − | NC | |
| 3412 | | | | |
| Morganella | Colourless | − | colourless | − |
| AR4080 | | | | |
| Pseudomonas | colourless | − | colourless | − |
| AR5196 | | | | |

NC: No bacterial growth.

Table I shows that the two media are capable of detecting pathogenic Yersinia enterocolitica but many species detected using the traditional CIN Agar medium are actually false positives, i.e. either non-pathogenic Yersinia enterocolitica strains or non-Yersinia enterocolitica, which is not the case with the medium according to this invention.

Surprisingly, a medium similar to the medium of the invention but in which indoxyl-beta-glucosaminide was replaced by indoxyl-beta-galactosaminide, another hexosaminidase substrate, does not allow colouring of pathogenic Yersinia enterocolitica strains. Thus, such a medium is incapable of differentiating pathogenic Yersinia enterocolitica from other bacteria, thus surprisingly demonstrating that only a glucosaminidine type substrate is capable of detecting pathogenic Yersinia enterocolitica strains with good specificity and good sensitivity.

### Example 2: Comparison of the Character of the Invention and the Beta-Galactosidase Character Formulas in g/l

Medium A according to the invention: Peptone 20, Sodium chloride 5, Agar 15, 5-bromo-6-chloro-3-indoxyl-N-acetyl-β-D-glucosaminide 0.1.

Medium B: Peptone 20, Sodium chloride 5, Agar 15, IPTG 0.04, 5-bromo 6-chloro 3-indoxyl-beta-galactoside 0.1.

Boxes are incubated for 24 h at 30° C.

Table II below shows the results:

TABLE II

| | Medium A | | Medium B | |
|---|---|---|---|---|
| | Colour | Detection | Colour | Detection |
| Y. enterocolitica | MAUVE | + | MAUVE | + |
| AR5580 | | | | |
| Citrobacter AR | colourless | − | MAUVE | + |
| 3030 | | | | |
| Klebsiella K3875 | colourless | − | MAUVE | + |
| E. coli AR 3740 | colourless | − | MAUVE | + |
| E. coli ATCC25922 | colourless | − | MAUVE | + |

The results presented in table II show that medium B is incapable of eliminating false positives, unlike the medium according to the invention.

Thus, the medium according to this invention has a better sensitivity and specificity, enabling easier detection in a single culture step without requiring a preliminary isolation step or a subsequent differentiation step. The consequence is that the associated costs are reduced and that better efficiency is obtained.

1. A culture medium for the detection of pathogenic Yersinia enterocolitica bacteria comprising:
nutrients necessary for growth of said bacteria to be detected, and
at least one chromogenic agent substrate of an acetyl-glucosaminidase.

2. The culture medium according to claim 1, wherein said chromogenic agent substrate of an acetyl-glucosaminidase is an indoxyl-beta-glucosaminide.

3. The culture medium according to claim 2, wherein said indoxyl-beta-glucosaminide is selected from the group consisting of 5-bromo-6-chloro-3-indoxyl-N-acetyl-beta-D-glucosaminide, and 6-chloro-3-indolyl-N-acetyl-beta-D-glucosaminide and 6-fluoro-indoxyl-N-acetyl-beta-D-glucosaminide.

4. The culture medium according to claim 1, further comprising a chromogenic agent substrate of beta-glucosidase.

5. The culture medium according to claim 4, wherein said chromogenic agent substrate of beta-glucosidase is 5-bromo-4-chloro-3-indolyl-beta-D-glucoside.

6. The culture medium according to claim 1, wherein said culture medium is a gelose culture medium.

7. The culture medium according to claim 1, wherein said culture medium comprises said chromogenic agent substrate of acetyl-glucosaminidase at a concentration of between 0.01 to 0.5 g/l.

8. The culture medium according to claim 4, wherein said culture medium comprises said chromogenic agent substrate of beta-glucosidase at a concentration of between 0.01 to 0.5 g/l.

9. The culture medium according to claim 1, wherein said culture medium comprises 5-chloro-2-(2,4)-dichlorophenoxy) phenol.

10. A method for direct detection of pathogenic Yersinia enterocolitica bacteria in a sample comprising the following successive steps:
a) inoculating said culture medium of claim 1, with said sample,
b) incubating said culture medium under conditions conducive to the growth of pathogenic Yersinia enterocolitica bacteria, and
c) detecting colonies formed on said culture medium corresponding to pathogenic Yersinia enterocolitica bacteria.

11. (canceled)

12. The culture medium according to claim 3, wherein said indoxyl-beta-glucosaminide is 5-bromo-6-chloro-3-indoxyl-N-acetyl-beta-D-glucosaminide.

13. The culture medium according to claim 1, wherein said culture medium comprises said chromogenic agent substrate of acetyl-glucosaminidase at a concentration of between 0.05 to 0.2 g/l.

14. The culture medium according to claim 4, wherein said culture medium comprises said chromogenic agent substrate of beta-glucosidase at a concentration of between 0.05 to 0.2 g/l.
